(19) United States
(12) Patent Application Publication
(10) Pub. No.: US 2007/0199566 A1
(43) Pub. Date: Aug. 30, 2007

(54) Respiratory apparatus

(76) Inventor: Eliezer Be'eri, Jerusalem (IL)

Correspondence Address: LAHIVE & COCKFIELD, LLP, ONE POST OFFICE SQUARE, BOSTON, MA, 02109-2127, US

(21) Appl. No.: 11/701,878

(22) Filed: Feb. 2, 2007

Related U.S. Application Data
(60) Provisional application No. 60/764,374, filed on Feb. 2, 2006.
(60) Provisional application No. 60/764,375, filed on Feb. 2, 2006.
(60) Provisional application No. 60/764,378, filed on Feb. 2, 2006.

(51) Int. Cl. A61M 16/00; A61B 5/08; A62B 7/00; F16K 31/02

(52) U.S. Cl. 128/204.23; 128/204.18; 600/529; 600/538

(57) ABSTRACT

Exemplary embodiments provide a respiratory device that can perform mechanical ventilation and/or inexsufflation. The respiratory device can include a mechanical medical ventilator, a sensor, a display and a processor. The mechanical medical ventilator assists a patient with the respiratory cycle. The sensor can measure an intra-thoracic respiratory parameter during the respiratory cycle. The display can display a graphical representation that dynamically depicts at least one of a patient's lung or thorax based on the intra-thoracic respiratory parameter in real-time during the respiratory cycle. The processor can update the graphical representation on the display in real-time based on the respiratory parameter. The processor updates the graphical representation to depict at least one of an expansion or a contraction of at least one of the lung or thorax during the respiratory cycle.

### RELATED APPLICATIONS

This application claims the benefit of U.S. Provisional Application No. 60/764,375, filed Feb. 2, 2006; U.S. Provisional Application No. 60/764,374, filed on Feb. 2, 2006; U.S. Patent Application No. 60/764,378, filed on Feb. 2, 2006; the disclosures of which are hereby incorporated in their entirety by this reference.

### FIELD OF THE INVENTION

The present invention relates to the field of respiratory devices. In particular, the present invention relates to an improved display technique that provides a real-time graphical representation of a patient's lungs and/or thorax based on a measured or sensed intra-thoracic respiratory parameter of a patient.

### BACKGROUND

Many types of respiratory apparatuses with displays are currently available. One type of respiratory apparatus is a mechanical ventilator. Another type of respiratory apparatus is an insufflation-exsufflation device (hereinafter an inexsufflator).

Mechanical ventilators are frequently used in the treatment of patients suffering from weak respiratory muscles and/or respiratory failure. A mechanical ventilator pumps air into the patients lungs under positive pressure and then allows for exhalation of that air to occur passively, driven by the natural elastic recoil of the patient's lungs; thereby assisting the patient with inspiration and/or expiration. In this manner, mechanical ventilators simulate a natural inhalation-exhalation respiratory cycle.

Inexsufflators pump air into the lungs under positive pressure (insufflation) and then actively suck the air out of the lungs under strong negative pressure (exsufflation). Inexsufflators are used to simulate a natural cough to remove secretions in the patient's lungs and air passages. For patients with a weak cough, inexsufflators can protect against infection by removing airway secretions from the lungs and air passages by assisting the patient with coughing.

For both types of devices (hereinafter “respiratory device(s)” unless otherwise noted) the operator of the device can manipulate the amount of air delivered by the device to the patient by controlling one or more airflow parameters, such as air pressure, air flow rate, volume of air delivered or time duration of the period of airflow.

These respiratory devices typically use conventional display techniques, such as an analogue manometer needle that rises and falls, a digital display of a bar that lengthens and shortens, or a line graph that rises and falls, to represent airway pressure changes. These display techniques may include gradations alongside the needle or the digital bar or graph can be provided as a pressure scale to indicate the pressure being generated within the airways. For example, a pressure scale of an inexsufflator typically runs from minus 100 cm H₂O through zero (atmospheric pressure) to plus 100 cm H₂O. As the device cycles from insufflation through to exsufflation, the manometer needle swings back and forth from positive to negative pressure readings on the pressure scale. An operator (e.g., patient or caregiver) of the respiratory device monitors the intra-thoracic air pressure generated by the respiratory device as it pumps air into or out of the lungs with each breath using the manometer to assess the functioning of the device. An accurate understanding of the intra-thoracic air pressure changes generated by the respiratory device is important because an intra-thoracic air pressure that is too high or too low may damage the patient's airways and/or upset the patient's physiology.

These conventional display techniques can make it difficult to determine the intra-thoracic pressure changes generated by an inexsufflator or ventilator, as the meaning of a rise or fall of a needle or a bar graph is not intuitive, especially to an unsophisticated observer who may not have an in-depth understanding of respiratory physiology. In many cases, these conventional display techniques can be confusing and misread by an operator creating a risk of harm to the patient. An understanding of the principles of respiratory physiology is typically required to accurately interpret the meaning of the needle's (or bar graph's) movement. For example, a swing of a manometer needle from a positive pressure reading to a negative pressure reading does not intuitively suggest that air is now being sucked out from the patient's lungs. The swing of a needle in a manometer (or change in a bar graph) generally only conveys meaningful information about how an inexsufflator is affecting the patient's body if the observer has been educated in the physiological meaning of the manometer (or bar graph). Today, many stable, chronically ventilated patients are cared for outside of intensive care units, for example in step-down geriatric facilities or even at home. In these environments it is common that family members or other caregivers who do not have formal or advanced medical/nursing training look after ventilated patients. For these caregivers, conventional measurement techniques are often confusing or meaningless. Even medical professionals (e.g., doctors, nurses, medical technicians, etc.) observing patients using these conventional unintuitive measurement techniques may not fully appreciate the meaning of the readouts that they see when they are rushed, distracted or tired, as commonly occurs in intensive care settings.

Using these conventional display techniques to depict airway-pressure changes can therefore be unhelpful and error prone for many patients or non-professional caregivers who have only a limited understanding of respiratory physiology. For these patients and caregivers, the manometers (or bar graphs) do not clearly inform them when the patient should breathe in deeply, and when they should start coughing if they wish to optimally coordinate their natural breathing cycle with that of mechanical medical respiratory apparatus. In addition, patients with caregivers who are not experienced at managing inexsufflators, or caregivers who have a low level of professional training, may fail to correctly interpret the pressure data provided by these conventional display techniques. For example, a caregiver may not appreciate that a negative-pressure reading on an inexsufflator means that air is being actively expelled from the patient's lungs.

There is, therefore, a need for an improved display technique that depicts the intra-thoracic respiratory parameters of a patient associated with the operation of mechanical medical respiratory apparatuses, such as ventilators and/or inexsufflators, in a manner that is intuitively understandable and that clearly informs an observer of about the status of these intra-thoracic respiratory parameters of a patient to reduce the risk of misinterpretations.

### SUMMARY

Exemplary embodiments provide an improved a graphical representation of the physiology of a respiratory cycle of a patient that is connected to a mechanical medical respiratory device, such as a ventilator and/or an inexsufflator. The graphical representation is responsive to one or more measured intra-thoracic respiratory parameters of a patient. Using at least one measured intra-thoracic respiratory parameter the graphical representation can expand and contract in real-time to imitate the actual expansion and contraction of the patient's lungs.

In one aspect a respiratory apparatus is disclosed. The respiratory apparatus includes a mechanical medical ventilator, a sensor, a display, and a processor. The mechanical medical ventilator assists a lung with a respiratory cycle. The sensor senses an intra-thoracic respiratory parameter during the respiratory cycle. The display displays a graphical representation that dynamically depicts at least one of a patient's lung or thorax based on the intra-thoracic respiratory parameter in real-time during the respiratory cycle. The processor updates the graphical representation on the display in real-time based on the intra-thoracic respiratory parameter. The processor updates the graphical representation to depict at least one of an expansion or a contraction of at least one of the lung or thorax during the respiratory cycle.

In another aspect a method of depicting a graphical representation of at least one of a lung or thorax that is based on a dynamic physiology of the patient's lungs is disclosed. The method includes sensing an intra-thoracic respiratory parameter generated by a medical mechanical ventilator during the respiratory cycle and displaying a graphical representation that dynamically depicts at least one of a patient's lung or thorax based on the intra-thoracic respiratory parameter in real-time during the respiratory cycle. The method also includes updating the graphical representation on the display in real-time based on the respiratory parameter. The processor updates the graphical representation to depict at least one of an expansion or a contraction of at least one of the lung or thorax during the respiratory cycle.

In yet another aspect, a medium for use on a computing system that holds computer-executable instructions for depicting a graphical representation of at least one of a lung or a thorax is disclosed. The instructions enable receiving an intra-thoracic respiratory parameter of a patient from a sensor associated with a mechanical medical ventilator during a respiratory cycle. The instructions also enable displaying a graphical representation that dynamically depicts at least one of a lung or thorax based on the intra-thoracic respiratory parameter that is received. The instructions further enable updating the graphical representation in real-time based on the respiratory parameter. The processor updates the graphical representation to depict at least one of an expansion or a contraction of at least one of the lung or thorax during the respiratory cycle.

### BRIEF DESCRIPTION OF THE FIGURES

The accompanying drawings, which are incorporated in and constitute a part of this specification, illustrate one or more exemplary embodiments and, together with the description, explain the invention. In the drawings,

FIG. 1A is a schematic diagram of one exemplary mechanical medical respiratory device;

FIG. 1B is a schematic diagram of the exemplary mechanical medical respiratory device of FIG. 1A using a different gate mechanism;

FIG. 1C is a schematic diagram of another exemplary mechanical medical respiratory device;

FIG. 1D depicts a distributed environment suitable for implementing exemplary embodiments of the present invention;

FIG. 2 illustrates a tubing suitable for use in the mechanical inexsufflation device of FIG. 1C;

FIGS. 3A-C depict an exemplary graphical representation of a patient's lungs to depict the real time physiology of the patient's lungs;

FIGS. 4A-B depict an exemplary animation that can be implemented in conjunction with exemplary embodiments of the graphical representation;

FIGS. 4C-D depict exemplary implementations for indicating a measured intra-thoracic respiratory parameter in relation to the graphical representation;

FIGS. 5A-B depict an exemplary graphical representation of a patient's thorax to depict the real time physiology of the patient's lungs;

FIGS. 6A-B depict a face of an exemplary control unit in accordance with exemplary embodiments of an exemplary mechanical medical respiratory device;

FIGS. 7A-B depict an alternative embodiment for displaying information and controlling an exemplary mechanical medical respiratory device;

FIG. 8A is a flow chart illustrating the steps involved in performing a mechanical inexsufflation using a mechanical inexsufflation device of an illustrative embodiment of the invention;

FIG. 8B is a flow chart illustrating an exemplary operation of depicting a graphical representation to imitate the actual physiology of a patient's lungs during ventilation;

FIG. 8C is a flow chart illustrating an exemplary operation of depicting a graphical representation to imitate the actual physiology of a patient's lungs during exsufflation;

FIG. 9 depicts an exemplary timing graphic that can be implemented as an alternative embodiment of the present invention; and

FIG. 10 is an alternative embodiment for locating a switch on a patient interface.

### DETAILED DESCRIPTION

Exemplary embodiments of the present invention provide an improved display technique that depicts a graphical representation of a patient's lungs and/or thorax to clearly convey various measured intra-thoracic respiratory parameters, such as air pressure, air volume, etc. The graphical representation is provided to improve understandability and clarity of various intra-thoracic respiratory parameters and to reduce potential misinterpretations that can result in harm to a patient.

The mechanical medical respiratory device can be, for example, a mechanical medical ventilator. The mechanical medical respiratory device can operate to insufflate a patients lungs using positive pressure. In some instance, the mechanical medical respiratory device can be an inexsufflator that may use negative airway pressure to exsufflate a patient's lung. The negative airway pressure can be forceful so as to simulate a cough.

The graphical representation can imitate the actual physiology of the patient's lungs and/or thorax in real time. In this manner, when inspiration occurs, the graphical representation expands and when the expiration occurs, the graphical representation contracts. The graphical representation can also be used to depict when a patient's lungs are forcefully exsufflated to simulate a cough. The graphical representation of the lungs and/or thorax can be anatomically correct.

The respiratory system is unique amongst all the internal organs of the body, inasmuch as changes in the internal physiology of the organ, i.e. an increase or decrease in intra-pulmonary air pressure or tidal volume, result in observable anatomical changes (i.e., expansion and contraction of the chest). Nonetheless, these observable anatomical changes do not convey a quantitative measurement of various intra-thoracic parameters.

In mechanically ventilated patients, it is often necessary to convey information relating to measured intra-thoracic respiratory parameters, such as increases and decreases in measured air pressure and tidal volume, to an observer. Embodiments of the present invention depict the physiological markers of inspiration and expiration as they relate to one or more intra-thoracic respiratory parameters, such as air pressure and/or tidal volume, using a graphical representation that shows a lung and/or chest expanding and/or contracting to facilitate the complex understanding of measured intra-thoracic parameters for all observers, including those with little or no formal knowledge of respiratory physiology.

The graphical representations therefore provide an intuitive approach to understanding the operation of the mechanical medical respiratory devices of the present invention. The graphical representations assist operators (who may be unfamiliar with the principals of respiratory physiology) in accurately interpreting the intra-thoracic respiratory parameter captured by the graphical representation. The graphical representations can be used to clearly inform a patient when they should breathe in deeply, and when they should start coughing.

In addition, exemplary embodiments provide quantitative data on a display. The quantitative data can provide various measurements that may be taken by the sensor or the other components associated with the mechanical medical respiratory device. For example, the quantitative data may represent a measured air pressure or air volume in a patient's lung.

There are a number of terms and phrases utilized herein that may require additional clarification. Such clarification is provided immediately below and throughout this disclosure

As used herein, the term “insufflation”, and the like, refers to the blowing of air, vapor, or a gas into the lungs of a patient.

As used herein, the term “exsufflation”, and the like, refers to the forced expiration of air, vapor or gas from the lungs of a patient.

As used herein, the term “real-time” refers to the updating of information at substantially the same rate the information is received. For example, exemplary embodiments discussed herein provide a processor for updating a display in real-time based on information received from a sensor. Real-time does not necessarily imply that there is no offset delay between when the information is received and when it is updated, but rather, may imply that some offset delay exists due to the causal relationship between the information received and the information being displayed. Such offset delays, however, are generally negligible and are often unobservable by an operator such that the display gives the impression that there is no delay between the patient's respiratory cycle and the depicted respiratory cycle.

As used herein, the term “sensor” refers to a device that senses and/or measures intra-thoracic respiratory parameters of a patient. As used herein, the terms “sense” and “measure” and their derivations are interchangeable.

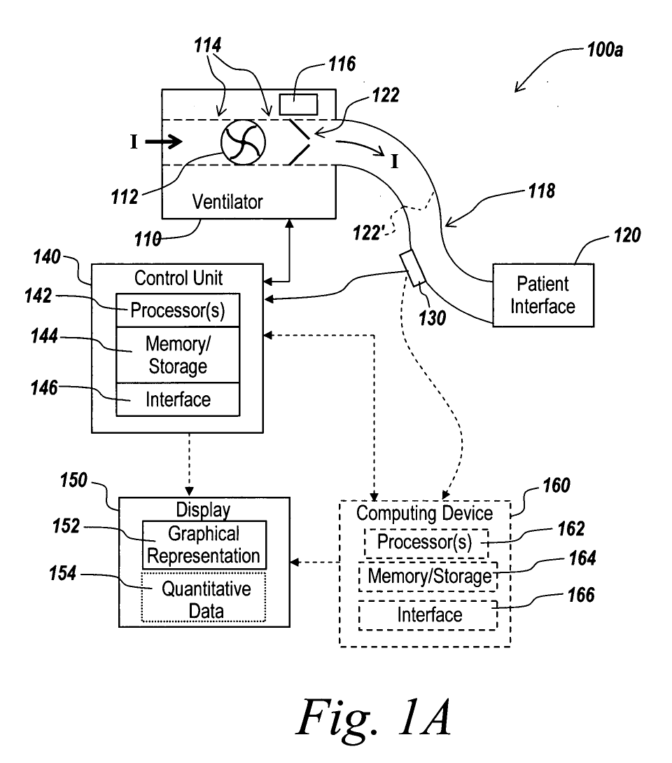

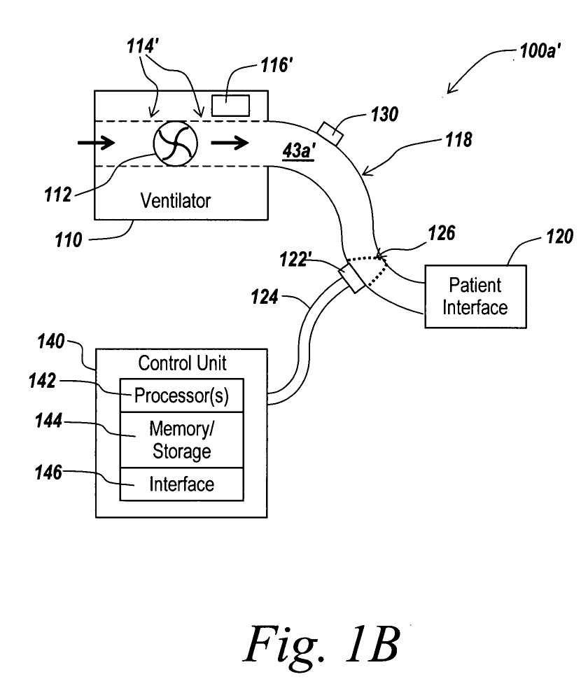

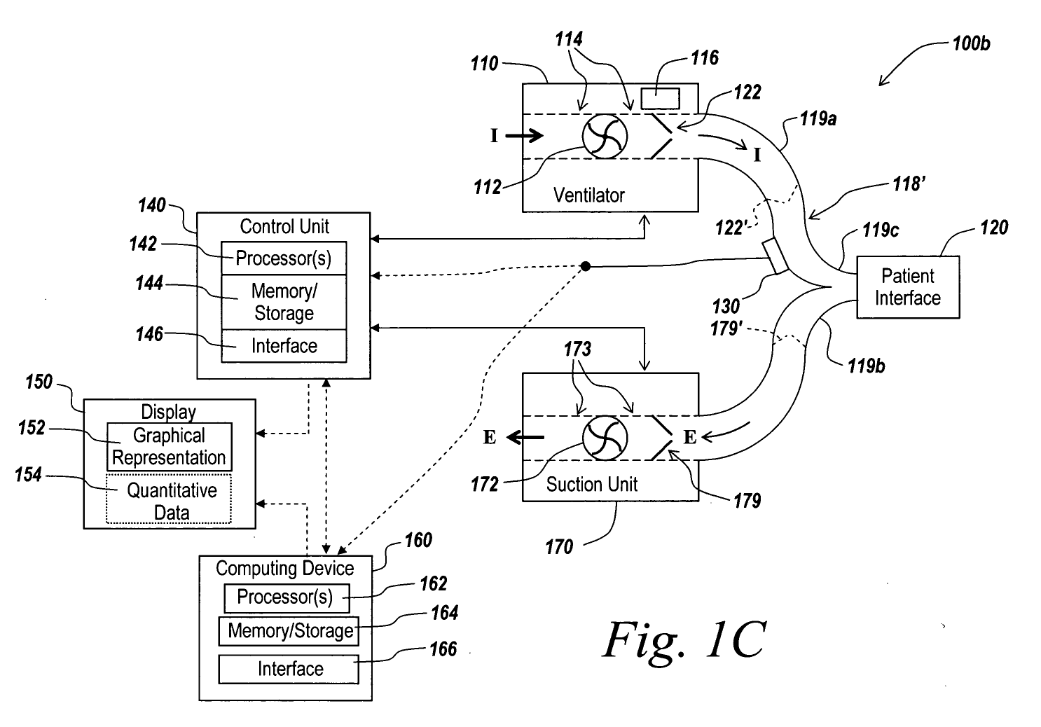

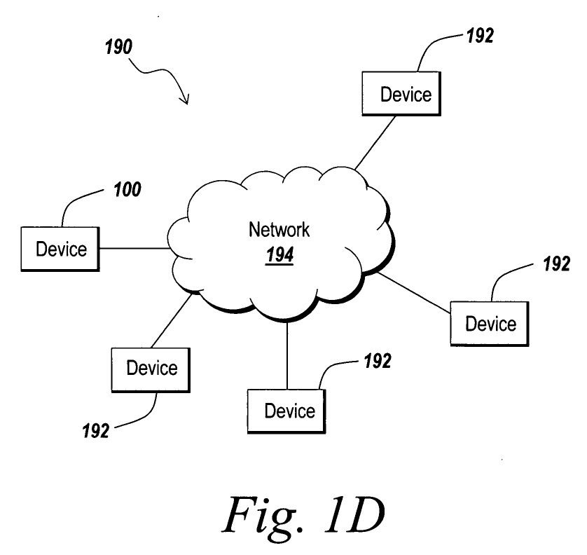

Referring to FIG. 1A, a mechanical medical respiratory device 100a (hereinafter device 100a) of one exemplary embodiment includes a mechanical medical ventilator 110 (hereinafter ventilator 110), a patient interface 120, a sensor 130, a control unit 140 and a display 150. The ventilator 110 is provided to generate airflow under positive-pressure. The positive pressure airflow may be used for insufflation of a patient. The illustrative ventilator 110 has a positive-pressure airflow generator 112 (hereinafter airflow generator 112), such as a turbine, piston, bellow or other devices known in the art, for generating airflow under positive pressure. One skilled in the art will recognize that the airflow generator 112 may be any suitable device or mechanism for generating positive pressure airflow and is not limited to the above-mentioned devices. An inflow airflow channel 114 (hereinafter channel 114) is connected to an inlet and outlet of the airflow generator 112 to convey and supply gas flow from the airflow generator 112. The direction of airflow through the airflow generator 112 and the associated airflow channel 114 is illustrated by the arrows labeled “I”.

The illustrative ventilator 110 for generating airflow under positive-pressure may be any suitable ventilator and is not limited to a particular type of medical ventilator. For example, the device may be a standard volume-cycled, flow-cycled, time-cycled or pressure-cycled life support or home use medical ventilator, or any medical ventilator or other device capable of generating positive end expiratory pressure (PEEP). Such devices are known in the art.

The ventilator 110 for generating airflow under positive-pressure preferably includes a calibration means 116 for calibrating the insufflatory airflow, as is standard practice in all medical ventilators. This calibration means 116 is also known as the “cycling mechanism” of the ventilator, and may operate on the basis of volume-cycled, flow-cycled, time-cycled or pressure-cycled mechanisms of calibration, or other basis known in the art.

The patient interface unit 120 interfaces the ventilator 110 with a patient. As shown, the inflow airflow channel 114 is connected to the patient interface unit 120 by means of tubing 118 or other suitable means. The illustrative patient interface unit 120 may be an endotrachael tube, a tracheostomy tube, a facemask or other suitable means known in the art for establishing an interface between a patient and another medical device, such as a ventilator or suction unit. The patient interface unit 120 is preferably of sufficient caliber to permit airflow at a flow rate that is substantially equivalent or in the range of the flow rate of a natural cough (generally corresponding to a flow rate of at least about 160 liters per minute through an endotracheal tube of internal diameter of about ten millimeters or about fourteen liters per minute through an endotracheal tube of about three millimeters internal diameter.) For example, the illustrative patient interface unit is configured to permit a negative pressure airflow therethrough of at least 14 liters per minute, ranging up to about 800 liters per minute, which covers the range of cough flow rates from infants to adults. The patient interface unit is also configured to permit positive pressure airflow from a mechanical medical ventilator.

Airflow channel 114 can include an optional valve, illustrated as by gate 122, for regulating airflow through the airflow channel 114. The gate 122 may selectively form a physical barrier to airflow within the airflow channel 114. The gate 122 may be selectively opened, to allow air to flow unobstructed through the airflow channel 114, or closed to block the airflow channel 114. For example, when the gate 122 is open, positive pressure airflow generated by the medical ventilator 110 is delivered to the patient interface unit via airflow channel 114 and tubing 118. In some embodiments, the gate 122 is not provided and the ventilator 110 can provide continuous, periodic, varying, etc., positive pressure flow into the patient's lungs using the positive airflow generator 112.

The gate 122 can comprise any suitable means for allowing reversible closing and opening of an airflow channel, including, but not limited to, membranes, balloons, plastic, metal or other mechanisms known in the art. For example, FIG. 1B depicts an alternative embodiment of a respiratory device 100a′ represented as a mechanical medical ventilator. In FIG. 1B, the gate 122′ within the inflow airflow path comprises a pneumatically-activated member, illustrated as a pneumatically-activated membrane 126. During operation of the device 100a′, the membrane gate 122′ is substantially flat and does not obstruct the lumen of the tubing 118. A pneumatic mechanism 124 is in communication with the membrane gate 122′. The control unit 140 controls the activation and deactivation of the membrane gate 122′. When the membrane gate 122′ is activated, the pneumatic mechanism 124 generates an increase in pneumatic pressure behind the membrane gate 122′, causing the membrane gate 122′ to bulge and thereby obstruct the lumen of the tubing 118, as illustrated by the dotted line 126. In an alternative embodiment, the gate 29′ may comprise a pneumatically activated piston, or any other pneumatically activated valve mechanism.

Referring to FIGS. 1A-B, the gate 122 (or gate 122′, hereinafter interchangeably referenced as the gate 122) or other valving means for selectively opening and closing the inflow airflow channel 114 can be located in any suitable position along the inflow airflow path. In one embodiment, the gate 122 can be located at any location along the inflow airflow channel 114 within the ventilator 110. The gate can be located at the air outlet of the ventilator 110 in the inflow airflow channel 114 or in another location. Alternatively, the gate 122 may be located within the tubing 118 and illustrated in phantom as gate 122′.

The device 100a (or device 100a′ hereinafter interchangeably referenced as the device 100a) can include a sensor 130, illustrated as a component on the tubing 118 between the patient interface 120 and a gate 122, for detecting one or more intra-thoracic respiratory parameter, such as an inspiratory pressure generated by the device 100a, particularly a peak inspiratory pressure, as described below. The sensor 130 can send data to various components of the device 100asuch that the device 100a can use the data to affect the operation of the device 100a. The sensor 130 can be located in any suitable location relative to the patient. For example, the sensor 10 may alternatively be located within the ventilator 110, within the patient interface 120, etc. The sensor 130 can be coupled, directly or indirectly, to various components, such as the control unit 140, the ventilator 110, the optional computing device 160, etc. The sensor 130 can measure the intra-thoracic respiratory parameters and can convert the parameters into an analog electrical signal. The analog electrical signal can be converted into a digital signal within the sensor 130, the ventilator 110, the control unit 140 or the computing device 160, or a separate component can be supplied, such as an analog-to-digital converter (ADC), that is coupled to an output of the sensor 130. In some embodiments, the analog electric signal can be used without converting it to a digital signal.

While FIGS. 1A-B depict a single sensor 130, one skilled in the art will recognize that the device 100a can have multiple sensors for sensing various intra-thoracic respiratory parameters of a patient. For example, a first sensor can be provided to sense an intra-thoracic pressure, while a second sensor can be provide to sense an intra-thoracic volume.

The control unit 140 interfaces with various components of the device 100a can include one or more microprocessor 142 (hereinafter processor 142), one or more memory and/or storage components 144 (hereinafter storage 144) and an interface 146. The processor 142 can run software and can control the operation of various components of the device 100a. The storage 144 can store instructions and/or data, and may provide the instructions and/or data to the processor 142 so that the processor 142 can operate various components of the device 100a. The control unit 140 can be an independent component, as depicted in FIG. 1A, or can be incorporated into another component of the device 100a, such as, for example, the ventilator 110.

The control unit 140 can receive and/or transmit information via the interface 146. The interface 146 can also include a hardware interface and/or software interface to allow an operator to interact with the device 100a. The information that is received and/or transmitted from the control unit 140 can be stored in the storage 144 and can be processed and or manipulated using the software algorithms running on the processor 142. Information can be received or transmitted to, for example, the ventilator 110, the sensor, 130, the display 150, etc. For example, one or more sensors, such as sensor 130, which may represent a pressure sensor, a flow sensor, etc., can send information to the control unit 140, via the interface 146, to be processed by processor 142. The interface 146 may also interface with a keyboard, a mouse, a microphone and/or other input devices and may be used to implement a distributed system via a network.

The control unit 140 may control electronic and mechanical functioning of the device 100a. For example, the control unit 140 may override the normal alarm functions of the ventilator 110 so as to prevent the alarms from sounding because of high pressure detected proximal to the closed gate 122. The control unit 140 may also or alternatively be programmed to initiate a cycle of mechanical medical ventilation to vary the positive pressure being forced into a patient's lungs. In another embodiment, the control unit 140 may adjust the timing of the inspiration and expiration cycles.

The control unit 140 may be located within the ventilator 110 or in any suitable location to effect control of various components of the device 100a. The control unit 140 can communicate with the ventilator unit 110 and/or the sensor 130 in either a wired or wireless manner.

The display 150 can interface with the control unit 140. The display 150 can be provided to assist with monitoring a patient who is connected to the device 100a. The display can depict a graphical representation 152 of the patient's lungs and/or thorax and can depict quantitative data 154 based on information processed by the control unit 140. For example, the control unit 140 can receive information from the sensor 130 that corresponds to one or more intra-thoracic respiratory parameters, and the processor can process the information. The processed information can be used to update a depiction of the graphical representation 152 and/or the quantitative data 154 on the display 150. In some embodiments the display 150 can be included in the control unit 140 such that the control unit 140 and the display 150 form a single component, while in other embodiments the display 150 can be a separate component that can receive data from the control unit 140.

Some embodiments of the device 100a can include a computing device 160 that interfaces with various components of the device 100a, such as, for example, the sensor 130, the control unit 140, the display 150, etc. The computing device 160 can include one or more processors 162 to run software to operate the computing device 160, one or more memory/storage components 164 that store code for the software and data to be used or that was generated by the processor, and an interface 166 that allows other device to interact with the computing device 160 and can be used to implement a distributed system. In one example, the control unit 140 may be used to control the operation of the device 100a, while the computing device 160 may be provided to receive information relating to the operation of the device 100a for processing or may receive an intra-thoracic respiratory parameter from the sensor 130. The computing device 160 may receive information directly from the sensor 130 to be processed and subsequently displayed on the display 150. In some embodiments that include the computing device 160, the control unit 140 may not be directly connected to the display, but rather may pass information to the computing device 160, which subsequently may depict the information on the display 150.

FIG. 1C depicts another exemplary embodiment of a mechanical medical respiratory device 100b (hereinafter device 100). In this example, the device 100b represents an inexsufflator. The device 100b includes the ventilator 110, the patient interface 120, the sensor 130, the control unit 140, the display 150, and a suction unit 170. The ventilator 110 is provided to generate airflow under positive-pressure, as described in FIGS. 1A-B.

The device 100b includes a suction unit 170 for generating airflow under negative pressure, which may be used to perform exsufflation of a patient. The illustrative suction unit 170 includes a negative-pressure airflow generator 172 (hereinafter airflow generator 172) for generating a suction force, and an outflow airflow channel 173 for conveying airflow to and through the airflow generator 172 under negative pressure. The pressure airflow generator 172 may be any suitable device or mechanism for generating negative pressure airflow, including, but not limited to, a turbine, piston, bellow or other devices known in the art. The direction of airflow to the airflow generator 172 and through the associated airflow channel 173 is illustrated by the arrows E.

The patient interface unit 120 interfaces the ventilator 110 and the suction unit 170 with a patient via tubing 118. As shown, the inflow airflow channel 112 and outflow airflow channel 173 are connected to the patient interface unit 120 by means of tubing 118′ or other suitable means. The illustrative patient interface unit 120 may be an endotracheal tube, a tracheostomy tube, a facemask or other suitable means known in the art for establishing an interface between a patient and another medical device, such as a ventilator 110 or suction unit 170. The patient interface unit 120 is preferably of sufficient caliber to permit airflow at a flow rate that is substantially equivalent or in the range of the flow rate of a natural cough (generally corresponding to a flow rate of at least about 160 liters per minute through an endotracheal tube of internal diameter of about ten millimeters or about fourteen liters per minute through an endotracheal tube of about three millimeters internal diameter.) For example, the illustrative patient interface unit is configured to permit a negative pressure airflow therethrough of at least 14 liters per minute, ranging up to about 800 liters per minute, which covers the range of cough flow rates from infants to adults. The patient interface unit is also configured to permit positive pressure airflow from a mechanical medical ventilator.

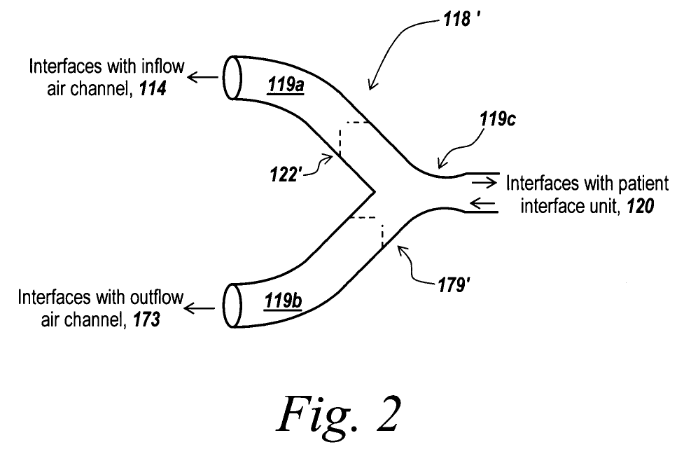

The illustrative tubing 118′, illustrated in detail FIG. 2, can be standard twenty-two millimeter diameter ventilator tubing or other suitable tubing known in the art. The tubing 118′ preferably is substantially branched, having two limbs 119a, 119b, each of which connects with air channels 114 and 173, respectively. The illustrative tubing 118′ is y-shaped, though the tubing 118′ may alternatively be t-shaped or have any other suitable shape known in the art. The ends of the limbs 119a and 119b may connect to and interface with the air channels 114 and 173 through any suitable means known in the art, such as friction fit and other connection means. The limbs 119a, 119b may extend at any suitable angle relative to a main portion 119c of the tubing 118′. As shown, the main portion 119c of the tubing 118′ connects to the patient interface 120 through any suitable means known in the art.

Alternatively, the tubing 118′ may comprise a single length of double-lumen tubing, with the two lumens joining together at the point of connection to the patient interface unit 120. One skilled in the art will recognize that any suitable means may be used for connecting both the ventilator 110 and the suction unit 170 to the patient interface unit 120. For example, two lengths of non-intersecting tubing coupled between the patent interface 120, the ventilator 110 and the suction unit 170.

Each airflow channel 114, 173 can include a valve, illustrated as gates 122, 179, respectively, for regulating airflow through the corresponding airflow channel. Each gate 122, 179 can selectively form a physical barrier to airflow within the corresponding airflow channel. Each gate 122, 179 may be selectively opened, to allow air to flow unobstructed through the corresponding airflow channel, or closed to block the corresponding airflow channel. For example, when gate 122 is open, positive pressure airflow generated by the ventilator 110 is delivered to the patient interface unit via channel 114 and tubing portions 119a, 119c. When gate 179 is open, negative pressure airflow generated by the suction unit 170 is permitted to flow from the patient interface device 120 to and through the suction unit 170 via tubing portions 119c, 119b and channel 173. The gates 122, 179 may comprise any suitable means for allowing reversible closing and opening of an airflow channel, including, but not limited to, membranes, balloons, plastic, metal or other mechanisms known in the art.

The inflow gate 122 or other valving means for selectively opening and closing the inflow airflow channel 114 can be located in any suitable position along the inflow airflow path. In one embodiment, the gate 122 may be located at any location along the inflow airflow channel 114 within the ventilator 110. The gate 122 can be located at the air outlet of the ventilator 110 in the inflow airflow channel 114 or in another location. Alternatively, the inflow gate 122 may be located within the tubing 118′, such as in the limb 119a and illustrated in phantom as inflow gate 122′. The outflow gate 179 is preferably located between the outflow airflow generator 172 and the patient interface unit 120. In one embodiment, the outflow gate 179 is located at the air inlet of the suction device 170. Alternatively the outflow gate 179 may be located in the tubing 118′, such as in the limb 119b. The alternative embodiment of the outflow gate 179′ is shown in phantom in FIGS. 1B and 2.

The device 100b can include the sensor 130, illustrated as a component on the tubing 118′ between the patient interface 120 and the gate 122, for detecting one or more intra-thoracic respiratory parameters, such as an inspiratory pressure generated by the device 10, particularly a peak inspiratory pressure, as described below. The sensor 130 can send data various components of the device 100b such that the device 100b can use the data to affect the operation of the device 100b. As with the device 100a, the sensor 130 in FIG. 1C can be located in any suitable location relative to the patient. For example, the sensor 130 may alternatively be located between the interface 120 and the gate 179, within the ventilator 110, within the patient interface 120, etc.

Some embodiments of the device 100b can include the computing device 160 that interfaces with various components of the device 100b, such as, for example, the sensor 130, the control unit 140, the display 150, etc. As discussed with reference to FIG. 1A, the computing device 160 can include one or more processors 162 to run software to operate the computing device 160, one or more memory/storage components 164 that store code for the software and data to be used or that was generated by the processor, and the interface 166 that allows other device to interact with the computing device 160 and can be used to implement a distributed system. In one example, the control unit 140 may be used to control the operation of the device 100b, while the computing device 160 may be provided to receive information relating to the operation of the device 100b for processing. The computing device 160 may also receive information directly from the sensor 130 to be processed and subsequently displayed on the display 150. In some embodiments that include the computing device 160, the control unit 140 may not be directly connected to the display, but rather may pass information to the computing device 160, which subsequently may depict the information on the display 150.

According to one embodiment, the device 100b can be formed by retrofitting the suction device 170 to the existing device 100a via the patient interface 120 and/or tubing 118 capable of selectively connecting both the suction unit 170 and ventilator 110 to the patient interface 120. Alternatively, a patient interface unit 120 with appropriate tubing 118′ may be provided for retrofitting a suction unit 170 and the ventilator 110 to perform mechanical inexsufflation.

FIG. 1D is an exemplary network environment 190 suitable for implementing distributed embodiments. The devices 100a and 100b are referred hereinafter to as device 100 such that the device 100 can represent either the device 100a, device 100a′, or the device 100b. The device 100 can be connected to other devices 192 via a communication network 194. The communication network 194 may include Internet, intranet, Local Area Network (LAN), Wide Area Network (WAN), Metropolitan Area Network (MAN), wireless network (e.g., using IEEE 802.11, IEEE 802.16, and/or Bluetooth), etc. In an exemplary implementation of network environment 190, device 100 can be connected to a patient and may gather information relating to the operation of the device 100 or relating to intra-thoracic respiratory parameters measured by the sensor 130. The device 100 can continuously send the information gathered by the device 100 over the communication network 190 to the other devices 192. The other device can receive the information and display in real-time a graphical representation of the patient's lungs and/or thorax as well as any quantitative data that is received. Using a distributed implementation can allow an operator to monitor the patient remotely by viewing the graphical representation of the patient's lungs and/or thorax so that an operator may not need to in the same geographical location as the device 100. This may be particularly important in an intensive car unit, a critical care unit, a “step down” unit or other medical care environments

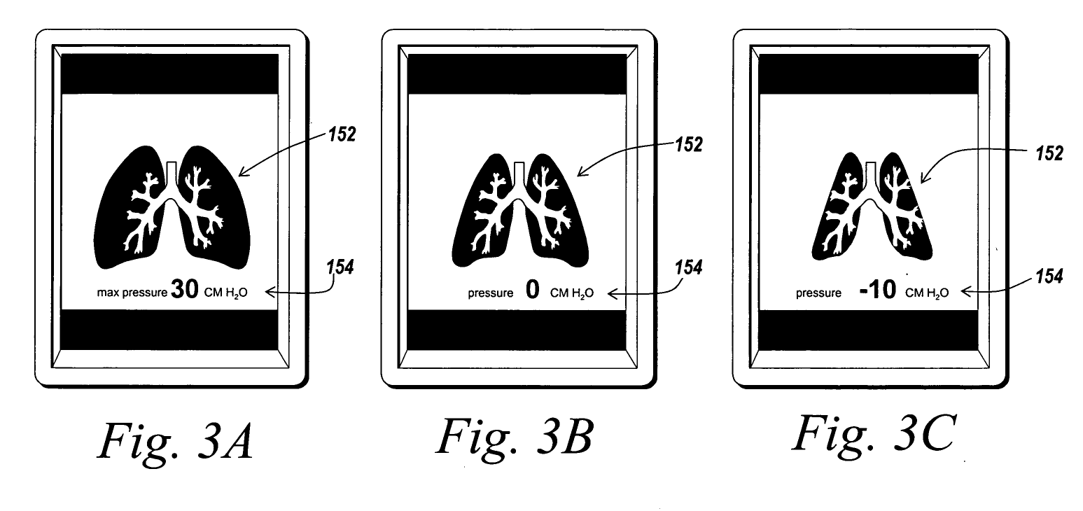

FIGS. 3A-C depict the exemplary display 150 of the graphical representation 152 of the lungs of a patient that is connected to the device 100 to depict the real time physiology of the patient's lungs as the device 100 operates from a users perspective. The graphical representation can be an anatomically correct depiction of the patient's lungs. The display can also include quantitative data 154 corresponding to one or more intra-thoracic respiratory parameters, which is discussed in more detail below.

The graphical representation 150 can depict the dynamic physiology of a patient's lungs based on the intra-thoracic respiratory parameters, such as airway-air pressure changes or volume changes, of the patient's lungs. The airway pressure or volume is depicted graphically, using the graphical representation 152, as a stylized silhouette of a human lung that changes size and color dynamically, in accordance with the dynamic changes in airway air-pressure or volume measured by the device 100. In one embodiment, as the device 100 operates to increase the airway pressure (i.e. positive values for airway pressure), the graphical representation 152 or the lung silhouette progressively increases in size. When the intra-thoracic respiratory parameter has reached a particular value, the size of the graphical representation 152 depicts the patient's lungs, as depicted in FIG. 3A. As the airway pressure decreases (in some cases due to the elastic recoil of the patient's lungs, but in other case due to exsufflation by the suction device 170), the size of the graphical representation 152 decreases. When the airway pressure is at atmospheric pressure, the graphical representation 152 is depicted as a lung silhouette of intermediate size, as depicted in FIG. 3B. When the operation of the device 100 causes the airway pressure to decrease to a negative airway pressure, the graphical representation 152 of the lung silhouette correspondingly decreases in size, as shown in FIG. 3C. The graphical representation 152 is updated in real time based on the measured airway pressure data such that the overall impression of the graphical representation 152 is one of a lung graphic moving smoothly and moving to correspond with the actual pressure changes occurring in the patient's lung.

The graphical representation 152, as described above, depicts the cyclic pressure changes of the respiratory cycle resulting from the operation of the device 100 using a recognizable graphic of a lung expanding and contracting where increasing positive pressure causes the graphical representation 152 of the lungs to expand, therefore, conveying to the observer that the lungs are being inflated by more air, while increasing negative pressure (i.e. decreasing positive pressure) causes the graphical representation 152 to contract, therefore conveying to the observer that the lungs are being deflated. As a result, the graphical representation 152 provides an intuitive depiction of the physiology of a patient's lungs so that an operator who is unfamiliar with the physiology of the lungs can clearly understand the function and operation of the device 100 as well as the various measurements that are taken using the device 100.

In some embodiments, the graphical representation 152 may use various colors to depict different stages of the respiratory cycle. For example, at atmospheric pressure the graphical representation 152 may use the color black, at a positive airway pressure the graphical representation may use the color green, and at a negative airway pressure the graphical representation may use the color red. In other embodiments, the graphical representation 152 can include an animation showing air going into and coming out of the patient's lungs.

Quantitative data 154 depicted in FIGS. 3A-C can be provided in addition to the graphical representation 152. The quantitative data 154 can correspond to data that is measured by the device 100, such as the instantaneous airway pressure, the instantaneous volume of gas (e.g., air) in a patient's lung(s), a peak airway pressure, a peak volume, etc.

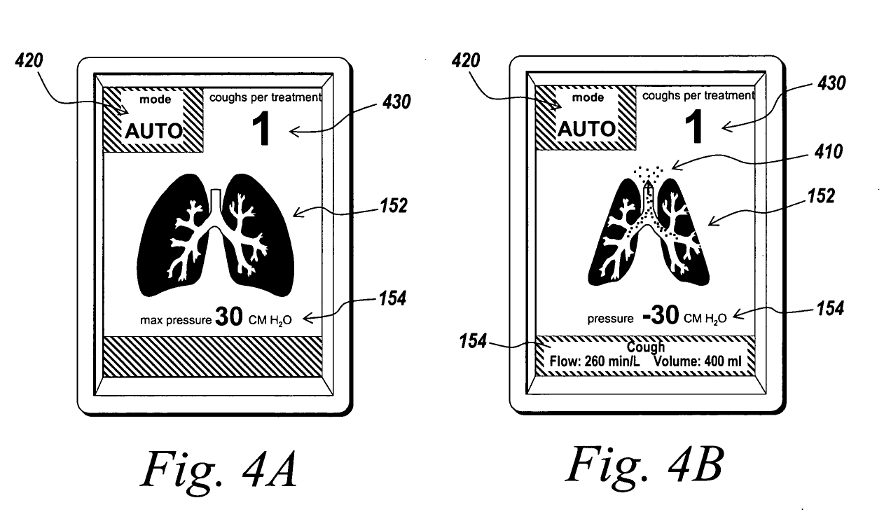

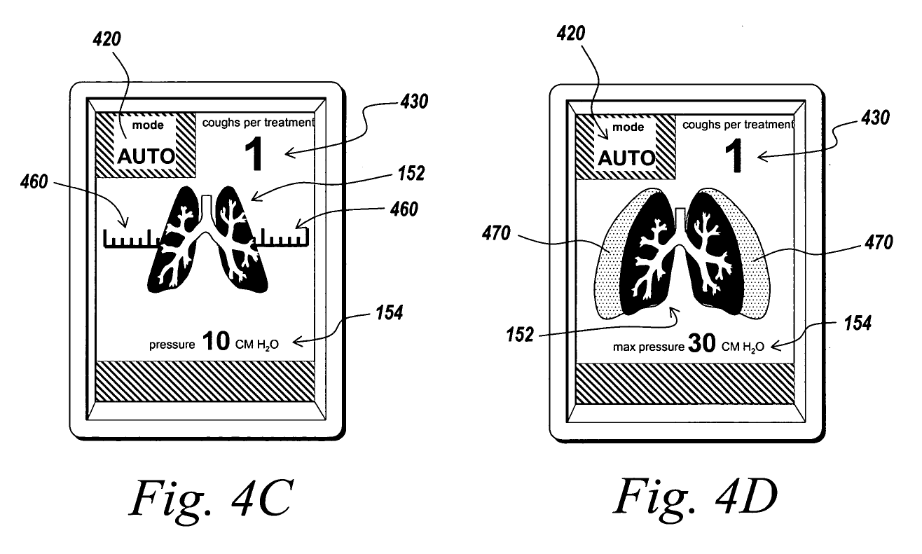

FIGS. 4A-B illustrates further exemplary depictions of the graphical representation 152 and the quantitative data 154 using the display 150 from a user's perspective. Again the graphical representation 154 represents the real-time physiology of a patient's lungs based on information from the one or more sensors 130. FIG. 4A depicts the lungs in the graphical representation 152 at full or near full capacity (e.g., pressure and/or volume) representing the insufflation of the patient's lungs via positive pressure from the ventilator 110. FIG. 4B depicts the lungs in the graphical representation 152 when the gate 122 is closed and the gate 179 is open, which results in a rapid deflation of the patient's lungs due the negative pressure generated by the suction unit 170 and represents the exsufflation of the patient's lungs via the suction unit 170. The graphical representation 152 decreases in size during the exsufflation to depict the reduction of pressure or volume in the patient's lungs. The graphical representation 152 in FIG. 4B can also include an animation of air and/or secretions 410 during the exsufflation of the patient's lungs. The animation of air and/or secretions 410 generally flows upwards and out of the lung silhouettes of the graphical representation 152. In other embodiments, an animation of air can be used to represent air being forced into the patient's lungs via positive pressure generated by the ventilator 110.

In further embodiments, a background set of line gradations 460, over which the lung silhouette expands and contracts can be used define the actual pressures depicted by the graphical representation at any point in time, as depicted in FIG. 4C.

In another preferred embodiment, a peak inspiratory pressure (PIP) attained by the graphical representation for each inspiratory cycle remains depicted on the screen as a lighter background shadow 470, while the graphical representation 152 contracts during exhalation and then re-expands again during the subsequent inspiratory cycle, as depicted in FIG. 4D. This feature enables the caregiver or patient to anticipate when the moment of PIP is next going to be reached. In this case, when the graphical representation 152 expands to reach the size of the lighter background shadow 470, the PIP is identified to the operator. The lighter background shadow 470 can allow an operator to determine an optimal moment for the patient to initiate exsufflation.

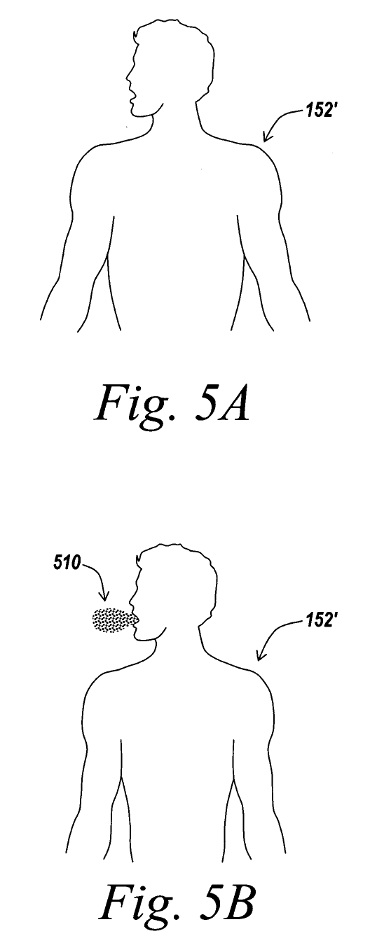

In other embodiments, a graphical representation 152′ can be depicted as a patient's thorax, as shown in FIGS. 5A-B. The graphical representation 152′ can represent the patients respiratory cycle. When the device 100 insufflates the patient's lungs, the graphical representation 152′ expands replicating the actual expansion of the patient's thorax, as shown in FIG. 5A. Similarly, when gas (e.g., air) is expelled from the patient's lung, either from the elastic recoil of the patient's lungs associated with the operation of device 100a and/or device 100b or from the forceful exsufflation of the patient's lungs associated with the operation of the device 100b, the graphical representation 152′ contracts to replicate the actual contraction of the patient's thorax, as shown in FIG. 5B. In some embodiments, the graphical representation 152′ can also include an animation of air and/or secretions 510 being expelled from the patient's lungs as the result of the exsufflation performed by the suction unit 170. In other embodiments, an animation of air can be used to represent air being forced into the patient's lungs via positive pressure generated by the ventilator 110 or air being expelled from the patient's lung either from the natural elastic recoil of the patient's lungs or from forceful exsufflation.

In other embodiments, the graphical representation 152 and the graphical representation 152′ can be combined to form a graphical representation that represents both the lungs and thorax of a patient.

When device 100 is implemented, the display 150 can depict a graphical user interface (GUI) that allows the user to set operational parameters 420 and 430. These parameters 420 and 430 can represent a mode of operation, a number of cough to generate per treatment, a pressure setting, a volume setting, etc. The user can adjust these parameters 420 and 430 via controls, which are discussed in more detail below.

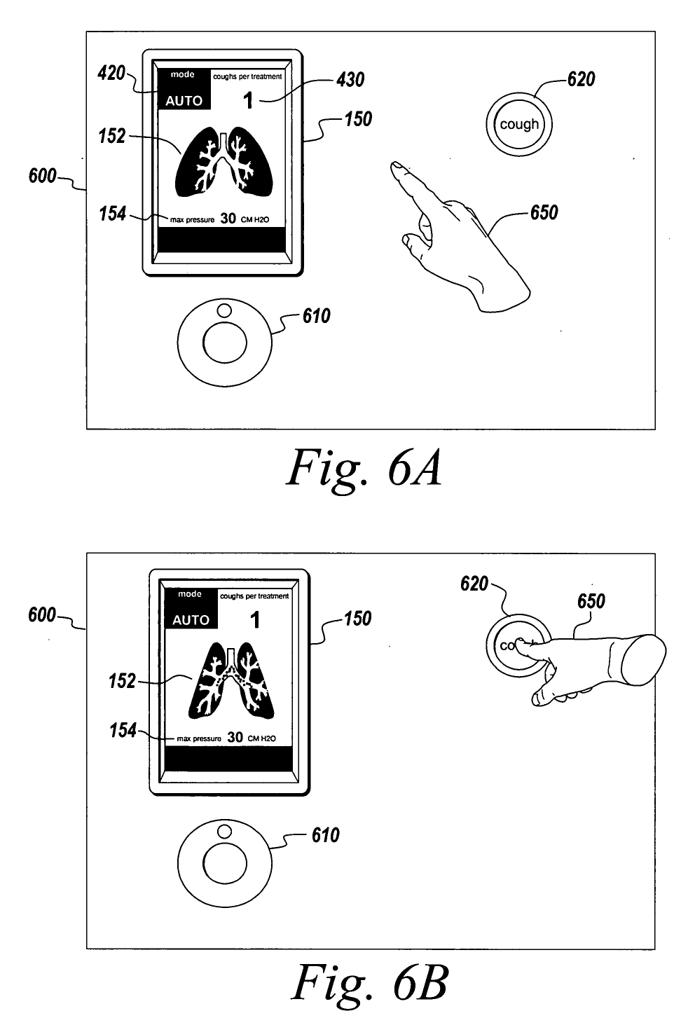

FIG. 6A depicts a user interface 600 of the control unit 140 in accordance with the exemplary embodiments of the device 100. The face 600 includes the display 150, a hardware control module 610 (hereinafter hardware control 610) and an optional hardware switch or button 620 (hereinafter switch 620). The display 150 can include the graphical representation 152, the optional quantitative data 154, and parameters 420 and 430, as discussed above with reference to FIGS. 3A-C and FIGS. 4A-B. While the display 150, as depicted in FIG. 6A is integrated into the control unit 140, one of ordinary skill in the art would recognize that the display 150 can be a separate component that interfaces with the control unit 140.

The hardware control 610 depicted in FIG. 6A represents a rotary control that can be rotated to adjust the values of parameters 420 and 430. The hardware control 610 can also incorporate a switch mechanism that allows an operator 650 to switch between available parameters that can be set. The hardware control 610 is an only one implementation of an input device that can be used in conjunction with the device 100 and is not meant to be limiting. Other implementations can be used to manipulate the parameters 420 and 430 as well as any other functions of the device 100. Some examples of other implementations can include, but are not limited to a key board, a mouse, a joy stick, a ball in a track, buttons, switches, etc.

The optional switch 620 may not be present on the device 100a, but may be present on the device 100b. The switch 620 can be used to initiate an exsufflation cycle of the device 100b. When the operator presses the switch 620, as shown in FIG. 6B, the gate 122 associated with the ventilator 110 is closed and the positive pressure of the ventilator ceases to insufflate the patient's lungs. Simultaneously with the closure of the gate 122, or shortly thereafter, the gate 179 opens and the suction unit creates a negative pressure to exsufflate the patient's lungs with sufficient force to simulate a cough. During exsufflation, the patient's lungs are rapidly deflated under the negative pressure created by the suction unit 170 simulating a cough with sufficient force to remove secretions from the patient's lungs and air passage. While the switch 620 is represented as a button, any type or form of switch can be used, such as a rocker switch, toggle switch, a proximity switch, an infrared switch, etc.

The gate 122 remains closed and the gate 179 remains open for a period of time after the switch is pressed. Once the period of time has elapsed, the gate 122 opens and the gate 179 closes and the ventilation of the patient continues.

In some embodiments, the device 100b can control the gates 122 and 179 automatically based on the information received from the sensor 130. The processor 142 and/or 162 can receive the information from the sensor 130 via the interface 146 and/or 166, respectively. For example, when the processor 142 and/or 162 receives information that corresponds to a patient who's lungs are fully or near fully insufflated, the device 100b can automatically close the gate 122 and open the gate 179 such that the patient's lungs are forcefully exsufflated; thereby removing secretions from the patient's lungs and/or air passage.

In the case where the device 100 is improperly connected or is not operating properly, an alert can be displayed on the display 150 or in another location to indicate to the operator 550 that there is an error. In addition to, or in the alternative of the alert that is displayed, the device 100 may generate an audio signal to indicate that an error has occurred.

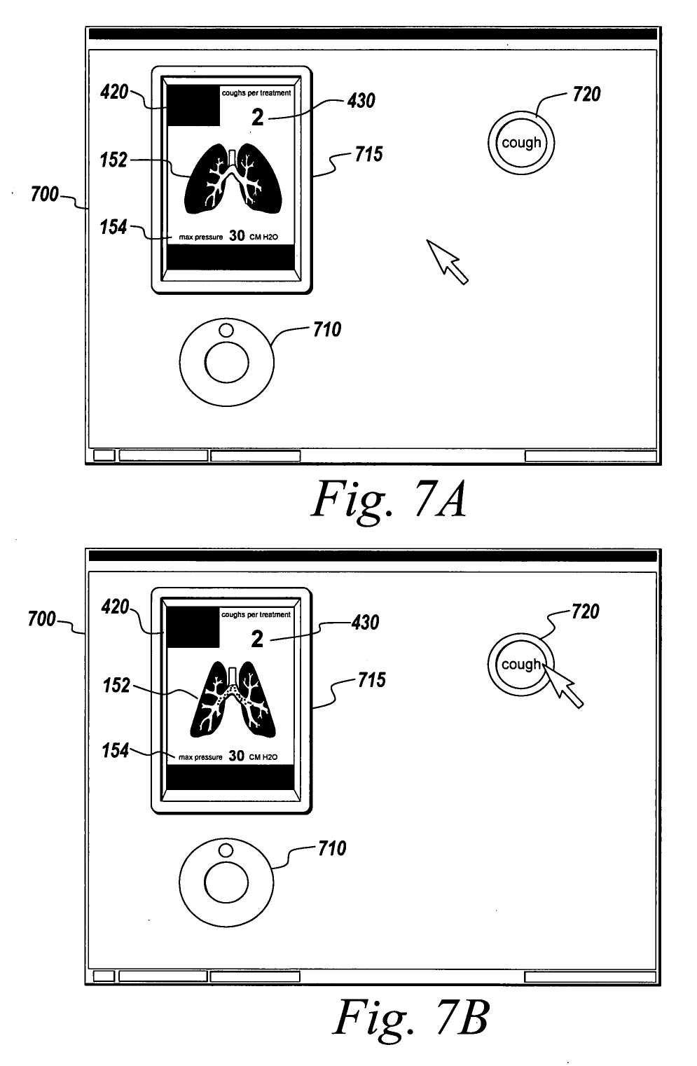

FIG. 7 depicts another embodiment for displaying information and controlling the device 100. In this example, the control unit 140 or the computing device 160 can implement a software interface 700 that can be displayed via display 150. The software interface 700 can operate in substantially the same manner as the hardware interface of FIGS. 6A-B and can include a software control 710, a display visualization 715 and a software switch 720.

The display visualization 715 can provide substantially the same information discussed with reference to FIGS. 3A-C, 4A-B, 5A-B and 6A-B. The software control 710 can be used to adjust or set the various parameters of the device 100 (e.g., parameters 420 and 430) and can take any form, such a graphical object that replicates control 610, a drop-down menu, a textual or graphical input area, etc. The software switch 720 can operate in substantially the same manner as the switch 620 in FIGS. 6A-B and can be represented in various forms, including but not limited to a graphical object that replicates a hardware switch, such as a push button switch, a rocker switch, a toggle switch, etc. When the user selects the software switch 720, the patient's lungs are exsufflated and the graphical representation 152 decreases in size, based on at least one measured intra-thoracic respiratory parameter, to represent the actual physiological contraction of the patient's lungs, as shown in FIG. 7B.

An operator can interface with the software interface using any suitable mechanism including, but not limited to a pointing device, such as a mouse; a data entry device, such as keyboard; a microphone; etc.

In some embodiments a combination of the user interface 600 and software interface 700 can be implemented. For example, in some embodiments the hardware switch 610 can be provided for switching from insufflation to exsufflation, while the software control 720 can be provided to manipulate various parameters of the device 100.

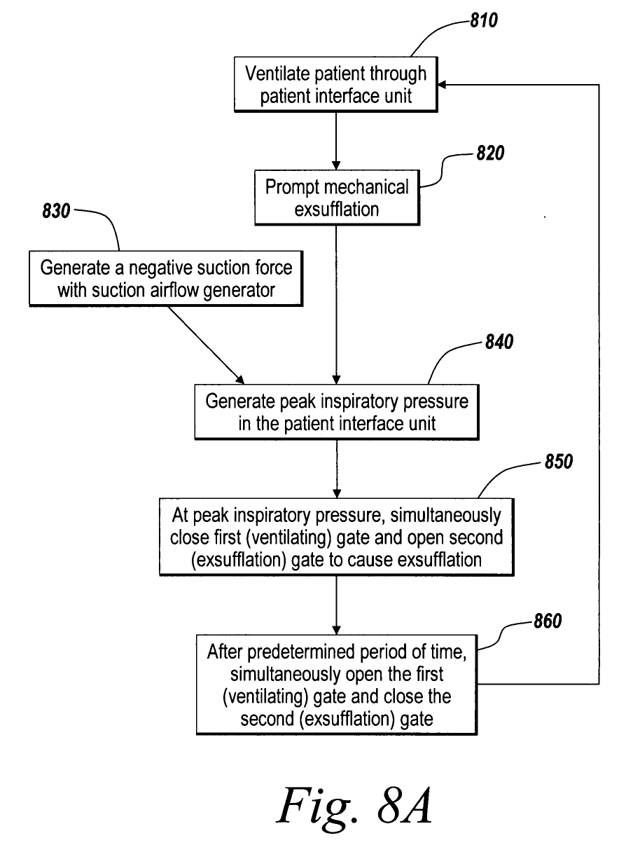

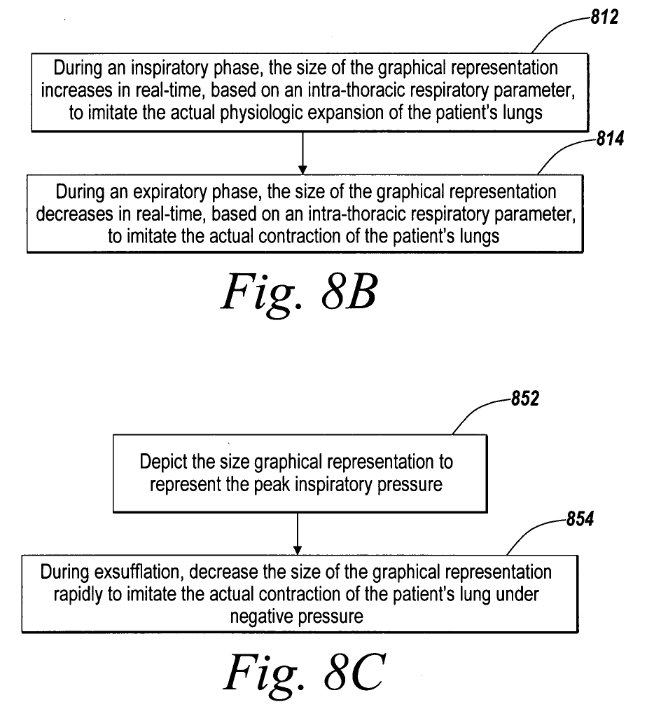

FIG. 8A is an exemplary flow diagram for operating the device 100b. In a first step 810, the device 100 is in a resting state, in which the ventilator 110 ventilates a patient through the patient interface unit 120. One skilled in the art would recognize that the ventilator 110 may require calibration or initialization prior to step 810 and that such calibration and initialization techniques are commonly known. Further one skilled in the art will recognize that in the case where device 100 represents devices 100a or 100a′, the step 810 represents the complete operation of the devices 100a and 100a′.

In the resting state, the first gate 122 in the inflow airpath, defined by airflow channel 114 and limb 119a, is open to allow positive pressure airflow generated by the generator 116 through the inflow airpath under positive pressure, while the second gate 179 in the outflow airpath, defined by outflow channel 173 and limb 119b is closed to prevent airflow through the outflow airpath. The device 100b remains in the first state, continuously ventilating the patient, until secretion removal by mechanical inexsufflation is desired or prompted.

When mechanical inexsufflation is prompted in step 820, the control unit 140 prepares to apply negative pressure airflow to the lungs to effect secretion removal. To effect secretion removal, the control unit 140 switches on, if not already on, the suction airflow generator 170 such that the suction airflow generator 172 then generates a negative suction force in step 830. Preferably, in step 830, the suction airflow generator produces a pressure differential of approximately 70 cm H₂O in comparison to the maximum pressure in the patient interface unit 120 during ongoing ventilation in step 820. Nevertheless, those skilled in the art will appreciate the suction airflow generator 172 produces a pressure differential of between about 30 to about 130 cm H₂O in comparison to the maximum pressure in the patient interface unit 120 during ongoing ventilation in step 820 and any value within this range may be suitable to permit inexsufflation of a patient. In one embodiment, the suction airflow generator 172 generates a suction force after mechanical inexsufflation is prompted in step 820. Alternatively, the suction airflow generator 172 may generate a negative pressure airflow even before prompting of the mechanical inexsufflation in step 820, such that suction force is in effect while or even before ventilation occurs in step 810. Steps 820 and 830 may be incorporated into a single step, involving powering on a suction unit 170 in preparation for performing secretion clearance, if the suction unit 170 is not already powered on.

To initiate mechanical inexsufflation, an operator can press or otherwise manipulate hardware switch 620 or software switch 720 on the hardware interface 600 or the software interface 700, respectively, or the control unit 140 can automatically initiate mechanical inexsufflation based on information received from the sensor 130, such as information relating to airway pressure. In other embodiments, a timing mechanism in the control unit 140 can be implemented such that inexsufflation is initiated periodically. During step 830, when the suction force is initiated, the outflow gate 179 remains closed, so that the patient interface unit 120 is not exposed to the suction force being generated. During step 830, positive pressure continues to be generated by the ventilator 110 simultaneously with the generation of negative pressure by the suction unit 170.

The conditions of step 830 continue until the ventilator 110 generates a peak inspiratory pressure in the patient interface unit 120 in step 840. The peak inspiratory pressure may be detected by the sensor 130, which then signals the control unit 140, or other suitable means. The use of a ventilator 110, which has means to measure and calibrate an insufflation, ensures that a patient's maximal lung vital capacity is reached, but not exceeded, to promote effective secretion removal.

When peak inspiratory pressure is reached, the control unit 140 can close the first, ventilating, gate 122 and opens the second, exsufflatory, gate 179 in step 850. In some embodiments, the closing of the first gate 122 and the opening of the second gate 179 occurs at substantially the same time. Switching between the gates 122 and 179 when both airflow generators 116 and 172 are operating rapidly suddenly exposes the patient to the pressure gradient generated by the suction airflow generator 172 and exsufflation of air from the lungs towards the suction unit 170 ensues. In an illustrative embodiment of the present invention, the simultaneous or near simultaneous closure of the first gate 122 ensures that the negative pressure generated by the suction airflow generator 172 does not suck atmospheric air in through the inflow airflow channel 114.

After a predetermined time period, which may be between about one and about two seconds or any suitable interval, the control unit 140, in step 860, causes the second, exsufflatory, gate 179 to close, and the first, ventilating, gate 122 to open. The closing of gate 179 and the opening of gate 122 can occur at substantially the same time. The suction unit 170 may be switched off after sealing the outflow airpath, or may continue to operate without affecting the subsequent ventilation by the ventilator 110.

Throughout steps 820 through 860, the ventilator 110 can operate continuously, including during the period of time that gate 122 is closed. Thus, immediately upon opening of gate 122, the patient is exposed to the ongoing positive pressure ventilation cycle of ventilator 110. The ventilator 110 then ventilates the patient through the patient interface unit 120 as in step 810, during a “pause” period until the control unit 140 initiates another mechanical inexsufflation cycle in step 820, and the illustrated steps 820-860 are repeated. During the pause period between mechanical inexsufflations, the patient receives full ventilation, according to all the ventilator's ventilation parameters (including provision of PEEP and enriched oxygen).

FIG. 8B depicts the operation of the display 150 in step 810. In step 812, as the ventilator 110 forces air into the patient's lung under positive pressure during an inspiratory phase, the size of the graphical representation 152 depicted via display 150 increases in real-time, based on an intra-thoracic respiratory parameter of the patient lungs measured by the sensor 130, to imitate the actual expansion of the patient's lungs. In step 814, as the patient's lungs expel the air (in some cases using the natural elastic recoil of the lungs) during an expiratory phase, the size of the graphical representation 152 depicted via the display 150 decreases in real-time, based on an intra-thoracic respiratory parameter of the patient's lungs measured by the sensor 130, to imitate the actual contraction of the patient's lungs. In some embodiments, an animation of air being forced into or out of the patient's lungs can be depicted with the graphical representation 152. FIG. 8B depicts the operation of the display in accordance with devices 100a, 100a′ and 100b.

FIG. 8C depicts operation of the display 150 in step 850 and is discussed in reference to device 100b (FIG. 1C). In step 852, the size graphical representation 152 depicted via the display 150 is increased to represent the peak inspiratory pressure. In step 854, when exsufflation occurs, the size of the graphical representation 152 depicted via the display 150 rapidly decreases to imitate the actual contraction of the patient's lung under negative pressure. As discussed herein, the graphical representation 152 of some embodiments can use animation to depict air being forced into and out of the patient's lungs. In some embodiments, the animation can be used to depict the removal of secretions from the patient's lungs and/or air passage.

Exemplary embodiments of the present invention do not require disconnecting the ventilated patient from his/her ventilator so as to perform inexsufflation. Therefore, the patient continues to receive essential ventilator parameters, such as PEEP provided by the ventilator, during the pause period between each inexsufflation cycle; thereby having the ability to facilitate secretion removal.

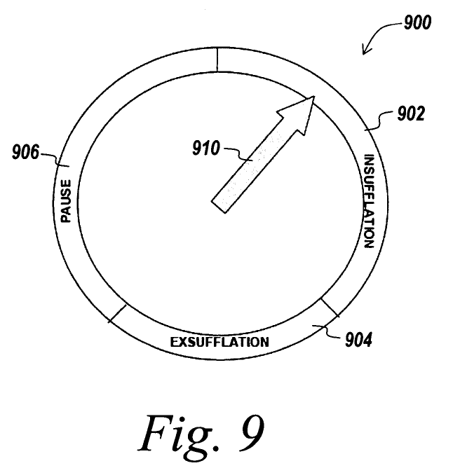

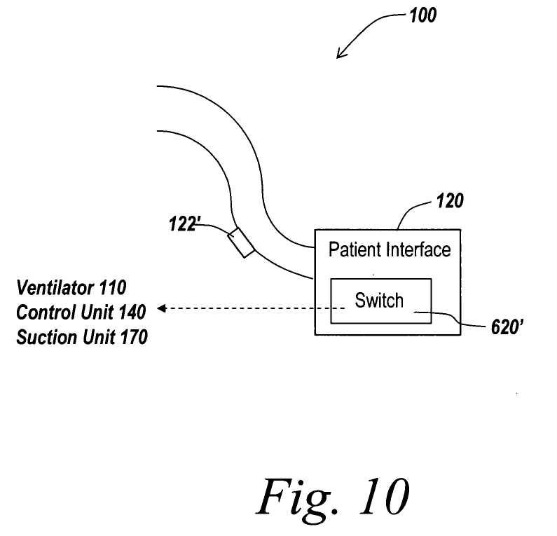

In an alternative embodiment, a timing graphic 900 that represents a timeline divided into three segments, where each segment represents phases of an inexsufflation cycle (insufflation 902, exsufflation 904 and pause 906) can be depicted on the display 150, as shown in FIG. 9. An indicator 910 can move along the timeline 900 such that the position of the indicator 910 informs the operator of the current phase and when the phase is going to transition into the next phase. The total length of the timeline can be fixed or adjusted by the operator via user interface 600 or software interface 700. The relative lengths of each of the three segments in relation to each other can also be variable, and can be calculated using software in the control unit 140 or the computing device 160. The indicator 910 moves along the timeline at a constant speed, traversing the entire timeline in the same time taken for the inexsufflator to complete one full automatic inexsufflation cycle (insufflation 902, exsufflation 904 and pause 906). As insufflation commences, the indicator 910 enters the “insufflation” segment 902 of the timeline, and then traverses that segment for the duration of the insufflation phase. Then, coincident with the inexsufflator switching to exsufflation, the indicator enters the “exsufflation” segment 904 of the timeline, and traverses that segment for the duration of that phase. Finally, the indicator traverses the “pause” segment 906 during the pause period of the inexsufflator's functioning. In alternative embodiments, the timing graphic may comprise only “insufflation” and “exsufflation” segments 902 and 904, without a segment representing the “pause” phase. In this embodiment, the indicator pauses between the two segments, or resets to the beginning of the “insufflation” segment and pauses there, during the actual “pause” phase of the inexsufflation cycle.

The timing graphic and indicator can be fashioned in any shape or form. In one embodiment, the timeline forms a whole circle, with each segment being an arc on the circumference of that circle, such that the point marking the end of the inexsufflation timeline is immediately adjacent to the point representing the beginning of the cycle, as shown in FIG. 9. In this embodiment, the indicator 910 may be a dot or bar that traverses the circumference of the circle, or an arrow with its origin at the center of the circle and its point on the circumference, similar to the hand of a watch sweeping around a watch face.

In an alternative embodiment, the timeline can be a straight line divided into segments, and when the indicator, in the form of a dot, square, triangle or any other shape, disappears at the end of the timeline, it instantaneously reappears at the beginning of the timeline, and continues to traverse the timeline.

In further embodiments, the timing graphic 900 may use different colors to represent each phase. For example, the color red may be used to represent the exsufflation phase, the color green may represent the insufflation phase and the color yellow may represent the pause phase.

Thus, by watching the progress of the indicator 910 as it moves along the timeline of the timing graphic 900, a patient using an automatic inexsufflator will be able to accurately anticipate the onset, duration and termination of each phase of the inexsufflator's automatic cycle.

In other embodiments, an audible signal, such as a voice counting down or a tone changing its pitch, may accompany the movement of the indicator and serve as an audio cue for the patient enabling the patient to anticipate the onset of the next phase in the inexsufflation cycle.

The timing graphic can also be used to depict the timing of respiratory cycles other than inexsufflation cycles, for example, the inhalation—exhalation cycle of a mechanical ventilator.

In an alternative embodiment, the switch 620′ can be located on the patient interface 120, as illustrated in FIG. 10. The switch 620′ can be located on one side of patient interface 120 and can be in communication with various components of the device 100, such as the ventilator 110, the control unit 140 and/or the suction unit 170. The sensor can be connected to the other components via a conductive wire, optical wire, or wirelessly. The switch 620′ can send a signal to, for example, the control unit 140 to switch between an insufflation phase and an exsufflation phase. Since the switch 620′ is located on the patient interface 120, it is possible for the operator to apply the patient interface 120 to the patient's face and operate the switch 620′ using a single hand. The switch 620′ may use an electric, hydraulic, pneumatic or any other mechanism to initiate an insufflation or exsufflation phase. In addition, switch 620′ may be configured as a push button, toggle switch, touch-pad, membrane or any other form of switch. The switch 620′ may be configured as a fixed component on the patient interface 120 or may be configured as a detachable element that can attach to be removed from the patient interface 120.

In an alternative embodiment, two or more control buttons or switches may be located on the patient interface 120, each controlling a different function of the device 100. For example, activating one switch may initiate insufflation, and releasing the button may terminate insufflation. Activating second switch may initiate and terminate exsufflation in a similar manner. When neither switch is activated, the device can enter a “pause” phase where neither positive nor negative pressure is being applied to the patient's lungs.

Locating the switch 620′ on the patient interface 120 greatly reduces the cumbersome nature of conventional inexsufflators. This is because conventional inexsufflators that are operated manually require two hands to operate effectively. One hand is required to hold the patient interface 120 to the patients face and the other hand is required to activate a switch that is located in another location.

When self-administering an inexsufflation treatment, many patients prefer to control the timing of these cycles manually, as the machine's automatic timing may not match the patient's natural breathing pattern well, resulting in respiratory discomfort and inefficient inexsufflation. Similarly, many caregivers prefer to administer inexsufflation treatments to patients in the manual mode rather than the automatic mode, so that they can ensure optimal timing of the treatment with the patient's respiratory pattern. Embodiments of the present invention, therefore, reduce the difficulty of self-administering inexsufflation treatments. Furthermore, embodiments alleviate the burden requiring a caregiver who wishes to administer a manual inexsufflation treatment to a patient to use two hands. As a result, the caregiver has a free hand thereby allowing the caregiver to perform chest physiotherapy on the patient at the same time as operating the inexsufflator.

The present invention has been described relative to certain illustrative embodiments. Since certain changes may be made in the above constructions without departing from the scope of the invention, it is intended that all matter contained in the above description or shown in the accompanying drawings be interpreted as illustrative and not in a limiting sense. It is also to be understood that the following claims are to cover all generic and specific features of the invention described herein, and all statements of the scope of the invention which, as a matter of language, might be said to fall therebetween.

Having described the invention, what is claimed as new and protected by Letters Patent is:

1. A respiratory apparatus comprising:
a mechanical medical ventilator for assisting a lung with a respiratory cycle;
a sensor for sensing an intra-thoracic respiratory parameter during the respiratory cycle;
a display for displaying a graphical representation that dynamically depicts at least one of a patient's lung or thorax based on the intra-thoracic respiratory parameter in real-time during the respiratory cycle; and
a processor for updating the graphical representation on the display in real-time based on the respiratory parameter, wherein the processor updates the graphical representation to depict at least one of an expansion or a contraction of at least one of the lung or thorax during the respiratory cycle.

2. The respiratory apparatus of claim 1, wherein the display further displays quantitative data depicting the sensed intra-thoracic respiratory parameter.

3. The respiratory apparatus of claim 1, wherein the mechanical medical ventilator is a positive pressure mechanical ventilator.

4. The respiratory apparatus of claim 1, wherein the mechanical medical ventilator is a negative pressure mechanical ventilator.

5. The respiratory apparatus of claim 1, wherein the graphical representation is anatomically correct.

6. The respiratory apparatus of claim 1, wherein the mechanical medical ventilator is an inexsufflator.

7. The respiratory apparatus of claim 1, wherein the sensed parameter is pressure.

8. The respiratory apparatus of claim 1, wherein the sensed parameter is volume.

9. The respiratory apparatus of claim 1, wherein the graphical representation further comprises an animation of air being expelled from at least one of the lungs or thorax.

10. The respiratory apparatus of claim 1, wherein the graphical representation further comprises an animation of air being insufflated into at least one of the lungs or thorax.

11. The respiratory apparatus of claim 1, wherein the graphical representation further comprises markers to identify a value of the intra-thoracic respiratory parameters, the value being identified based on a position of the graphical representation in relation to one of the markers.

12. The respiratory apparatus of claim 1, wherein the graphical representation further comprises a shaded background to indicate a peak intra-thoracic respiratory parameter that is measured during the respiratory cycle.

13. The respiratory apparatus of claim 1, wherein the display provides a graphical user interface (GUI).

14. A method of depicting a graphical representation of at least one of a lung or thorax that is based on a dynamic physiology of the patient's lungs, the method comprising:
sensing an intra-thoracic respiratory parameter generated by a medical mechanical ventilator during the respiratory cycle;
displaying a graphical representation that dynamically depicts at least one of a patient's lung or thorax based on the intra-thoracic respiratory parameter in real-time during the respiratory cycle; and
updating the graphical representation on the display in real-time based on the respiratory parameter, wherein the processor updates the graphical representation to depict at least one of an expansion or a contraction of at least one of the lung or thorax during the respiratory cycle.

15. The method of claim 14, wherein the displaying further displays quantitative data depicting the sensed intra-thoracic respiratory parameter.

16. The method of claim 14, wherein the mechanical medical ventilator is a positive pressure mechanical ventilator.

17. The method of claim 14, wherein the mechanical medical ventilator is a negative pressure mechanical ventilator.

18. The method of claim 14, wherein the graphical representation is anatomically correct.

19. The method of claim 14, wherein the mechanical medical ventilator is an inexsufflator.

20. The method of claim 14, wherein the intra-thoracic respiratory parameter is pressure.

21. The method of claim 14, wherein the intra-thoracic respiratory parameter is volume.

22. The method of claim 14, wherein the graphical representation further comprises an animation of air being expelled from at least one of the lungs or thorax.

23. The method of claim 14, wherein the graphical representation further comprises an animation of air being insufflated into at least one of the lungs or thorax.

24. The method of claim 14, wherein the graphical representation further comprises markers to identify a value of the intra-thoracic respiratory parameters, the value being identified based on a position of the graphical representation in relation to one of the markers.

25. The method of claim 14, wherein the graphical representation further comprises a shaded background to indicate a peak intra-thoracic respiratory parameter that is measured during the respiratory cycle.

26. The method of claim 14, wherein the displaying comprises a graphical user interface (GUI).

27. A medium for use on a computing system, the medium holding computer-executable instructions for depicting a graphical representation of at least one of a lung or a thorax, the medium comprising instructions for performing the method of:
receiving an intra-thoracic respiratory parameter of a patient from a sensor associated with a mechanical medical ventilator during a respiratory cycle;
displaying a graphical representation that dynamically depicts at least one of a lung or thorax based on the intra-thoracic respiratory parameter that is received; and
updating the graphical representation in real-time based on the respiratory parameter, wherein the processor updates the graphical representation to depict at least one of an expansion or a contraction of at least one of the lung or thorax during the respiratory cycle.

28. The medium of claim 27, wherein the displaying further displays quantitative data depicting the intra-thoracic respiratory parameter.

29. The medium of claim 28, wherein the intra-thoracic respiratory parameter is at least one of a peak inspiratory pressure, an instantaneous pressure, an instantaneous volume, or a peak volume.

30. The medium of claim 27, wherein the mechanical medical ventilator is a positive pressure mechanical ventilator.

31. The medium of claim 27, wherein the mechanical medical ventilator is a negative pressure mechanical ventilator.

32. The medium of claim 27, wherein the graphical representation is anatomically correct.

33. The medium of claim 27, wherein the mechanical medical ventilator is an inexsufflator.

34. The medium of claim 27, wherein the intra-thoracic respiratory parameter is pressure.

35. The medium of claim 27, wherein the intra-thoracic respiratory parameter is volume.

36. The medium of claim 27, wherein the graphical representation further comprises an animation of air being expelled from at least one of the lungs or thorax.

37. The medium of claim 27, wherein the graphical representation further comprises an animation of air being insufflated into at least one of the lungs or thorax.

38. The medium of claim 27, wherein the graphical representation further comprises markers to identify a value of the intra-thoracic respiratory parameters, the value being identified based on a position of the graphical representation in relation to one of the markers.

39. The medium of claim 27, wherein the graphical representation further comprises a shaded background to indicate a peak intra-thoracic respiratory parameter that is measured during the respiratory cycle.

40. The medium of claim 27, wherein the displaying comprises a graphical user interface (GUI).

41. An inexsufflator for cough simulation to remove broncho-pulmonary secretions of a patient comprising:
a patient interface unit having a switch mounted thereto to selectively couple said patient interface unit with a port of a medical inexsufflator,
wherein activation of the switch from a first position to a second position at or near lung capacity of the patient initiates exsufflation of the lung to simulate a cough.
